(19) United States
(12) Patent Application Publication
(10) Pub. No.: US 2026/0188454 A1
(43) Pub. Date: Jul. 2, 2026

(54) REMOTE SUPERVISED PREPARATION, VERIFICATION, AND CONTROLLED DISPENSING OF MEDICATION DOSES

(71) Applicant: Opio Connect, Inc., Kalispell, MT (US)

(72) Inventors: Amber Norbeck, Kalispell, MT (US); Michael Dawson, Kalispell, MT (US); Kelly Chu, Kalispell, MT (US); Thomas Hoffmann, Kalispell, MT (US)

(21) Appl. No.: 19/543,037

(22) Filed: Feb. 18, 2026

Related U.S. Application Data
(63) Continuation-in-part of application No. 19/306,568, filed on Aug. 21, 2025.
Application No. 19/306,568 is a continuation-in-part of application No. 19/208,950, filed on May 15, 2025, now abandoned.
Application No. 19/306,568 is a continuation-in-part of application No. 19/211,700, filed on May 19, 2025, now abandoned.
(60) Provisional application No. 63/647,719, filed on May 15, 2024.
(60) Provisional application No. 63/648,895, filed on May 17, 2024.

(51) Int. Cl. G16H 20/13; G06F 21/32; G16H 40/67; G16H 80/00

(52) U.S. Cl. CPC G16H 20/13; G06F 21/32; G16H 40/67; G16H 80/00

(57) ABSTRACT

A system and method are provided for remotely supervised preparation, verification, and controlled dispensing of medication doses at geographically distributed remote dispensing units. A recipient and a supervisor are authenticated. A real-time audiovisual session is established between the recipient and at least the supervisor, and additional authenticated participants may join without authority to actuate dose release. The system receives an electronic medication order and corresponding label data originating from an external clinical information system and authorizes preparation of doses at the remote dispensing unit based on the order. Prior to any release, verification data is presented to the supervisor. A controlled mechanical transfer mechanism is actuated to release a prepared dose to the recipient only after verification and affirmative authorization by an authenticated supervisor. The disclosed systems and methods are applicable to controlled substances and other medications requiring supervised dispensing.

### CROSS-REFERENCE TO RELATED APPLICATIONS

This application is a continuation-in-part and claims benefit of U.S. patent application Ser. No. 19/306,568 filed Aug. 21, 2025, which is a continuation-in-part and claims benefit of U.S. patent application Ser. No. 19/208,950 filed May 15, 2025, which is a non-provisional and claims benefit of U.S. Provisional Application No. 63/647,719 filed May 15, 2024, the specification(s) of which are incorporated herein in their entirety by reference.

U.S. patent application Ser. No. 19/306,568 is also a continuation-in-part and claims benefit of U.S. patent application Ser. No. 19/211,700 filed May 19, 2025, which is a non-provisional and claims benefit of U.S. Provisional Application No. 63/648,895 filed May 17, 2024, the specification(s) of which are incorporated herein in their entirety by reference.

### FIELD OF THE INVENTION

The present invention relates to systems and methods for remotely supervised preparation, verification, and controlled mechanical dispensing of medication doses, including via biometric authentication, role-based supervisory actuation control, multi-source verification data capture, and integration with one or more external clinical information systems.

### BACKGROUND OF THE INVENTION

For many underserved and geographically remote communities, it is difficult to provide timely access to medications that require supervised dispensing, controlled release, identity verification, and auditability. In many settings, applicable laws, regulations, payer requirements, or clinical protocols require that only authorized clinicians may authorize and/or dispense certain medications and may require real-time oversight of dispensing events. In lower-population regions, these requirements can force recipients to travel long distances to centralized facilities, creating barriers to care and operational burden.

Although telehealth platforms can support remote clinical encounters, telehealth alone does not address the need for secure, supervised, and verifiable preparation and controlled release of medication doses at distributed locations. Existing telehealth workflows generally do not integrate secure, role-segmented mechanical control of medication preparation and release at geographically distributed dispensing units with real-time supervisory verification, biometric authentication of both recipients and supervisory personnel, and comprehensive transaction audit logging. By way of example, these challenges may be particularly acute for medication workflows that require enhanced oversight, including certain controlled substances; however, the need extends to other medication categories that benefit from supervised dispensing and verifiable release.

### BRIEF SUMMARY OF THE INVENTION

It is an objective of the present invention to provide methods and systems that enable remotely supervised preparation, verification, and controlled mechanical dispensing of medication doses, including but not limited to methadone, buprenorphine, and other controlled or non-controlled therapeutic agents, as specified in the independent claims. Embodiments of the invention are set forth in the dependent claims. Embodiments of the present invention may be freely combined with one another unless mutually exclusive.

In certain embodiments, a supervisory user authenticated using at least (i) a personal identification credential and (ii) a biometric identifier is granted exclusive authority to issue actuation commands for mechanical operations including preparation initiation, cancellation, quarantine, locking, unlocking, and controlled release of one or more medication doses. Additional session participants, including physicians, pharmacists, counselors, schedulers, or other authorized personnel, may join real-time audiovisual sessions and may view verification data without permission to issue actuation commands or otherwise actuate a mechanical release mechanism.

In certain embodiments, each remote dispensing unit includes a physical isolation structure disposed between a secure preparation compartment and a recipient interaction compartment. The physical isolation structure may comprise an internal isolation gate, barrier, or anti-reach mechanism configured to prevent insertion of a recipient's hand or foreign object into the secure preparation compartment while permitting controlled transfer of a prepared medication container to a recipient-accessible transfer region only when actuated under supervisory authorization.

In certain embodiments, the present invention features a method for remotely supervising preparation and controlled dispensing of medication doses to a recipient. The method may comprise determining an identity of the recipient using a biometric sensing component; establishing remote audiovisual communication between the recipient and a supervisory user; receiving an order and corresponding label data transmitted from an external clinical information system; authorizing mechanical preparation of one or more medication doses based on the received order; capturing verification data associated with the prepared medication dose, including preparation telemetry, digital label data, and one or more images of a prepared medication container; and, upon supervisory authorization, actuating a controlled mechanical transfer mechanism to release the medication dose to the recipient while maintaining physical isolation from a secure preparation compartment. The method may further comprise generating timestamped audit records associated with identity verification, preparation steps, supervisory actions, and release outcomes.

In certain embodiments, verification data includes simultaneous visual feeds from a plurality of cameras directed toward a recipient interaction area, a preparation area, and a transfer mechanism to enable real-time supervisory confirmation

In certain embodiments, a single supervisory user may concurrently supervise preparation and dispensing events across a plurality of geographically distributed remote dispensing units.

In certain embodiments, prepared medication containers may be automatically diverted into a secured quarantine compartment based on supervisory input, verification discrepancies, or system-detected conditions.

In certain embodiments, the present invention further features a computing system configured to supervise preparation and controlled dispensing of medication doses. The system may comprise a communication component configured to communicatively couple to a plurality of remote dispensing units, a processor, and a memory component comprising computer-readable instructions. The instructions may comprise accepting biometric data from a remote dispensing unit; confirming an identity of a recipient based on the biometric data; receiving order and label data from an external clinical information system; establishing audiovisual communication between the supervisory user and the recipient through the remote dispensing unit; capturing and presenting verification data; and transmitting supervisory control signals that actuate mechanical preparation, quarantine, locking, unlocking, or controlled release of medication doses.

Any feature or combination of features described herein is included within the scope of the present invention provided that the features included in any such combination are not mutually inconsistent, as will be apparent from the context of this specification and the knowledge of one of ordinary skill in the art. Additional advantages and aspects of the present invention are apparent in the following detailed description and claims.

### BRIEF DESCRIPTION OF THE SEVERAL VIEWS OF THE DRAWING

The features and advantages of the present invention will become apparent from a consideration of the following detailed description presented in connection with the accompanying drawings in which:

FIG. 1A shows a flow diagram of a method for remotely supervised preparation, verification, and controlled release of medication doses.

FIG. 1B shows an alternative flow diagram including multi-participant sessions, quarantine/cancellation controls, and concurrent supervision.

FIG. 2A shows a block diagram of a distributed system including a supervisory computing platform and multiple remote dispensing units.

FIG. 2B shows a diagram of supervisory authentication, verification inputs, and mechanical actuation of the present invention.

FIG. 2C shows the remote dispensing unit showing internal robotic automation components.

FIG. 3A SHOWS THE SUPERVISORY INTERFACE SHOWING VERIFICATION DATA AND RELEASE controls.

FIG. 3B shows a recipient biometric match and pre-session verification.

FIG. 3C shows an order receipt and preparation of authorization controls.

FIG. 3D shows the final verification data and conditional release/quarantine controls.

FIGS. 3E-3F shows the supervisory dual-factor authentication prior to release.

FIG. 3G shows the recipient biometric re-authentication immediately prior to release.

FIG. 3H shows the recipient acknowledgment and electronic signature process of the present invention.

FIGS. 3I-3J show the internal isolation gate positioned between a secure preparation compartment and a recipient interaction compartment preventing recipient access to internal robotic components.

FIG. 3K shows the remote medication dispensing station with integrated telehealth interface and overhead camera.

FIG. 4A shows the controlled transfer door in an unlocked state enabling recipient retrieval of a prepared medication dose.

FIG. 4B shows recipient retrieval of medication through a controlled transfer mechanism under live audiovisual supervision.

FIGS. 5A-5C show the automated quarantine mechanism showing robotic removal of prepared medication containers from an intermediate buffer and transfer into a secured quarantine compartment.

FIG. 6 shows a multi-camera supervisory monitoring interface displaying simultaneous recipient, preparation, and transfer-area video feeds.

FIG. 7A shows a photograph of the user interface implemented in the system for remotely approving and dispensing medications directly to a patient for opioid addiction treatment of the present invention.

FIG. 7B shows a photograph of the user interface and screen implemented in the system for remotely approving and dispensing medications directly to a patient for opioid addiction treatment of the present invention.

FIGS. 8A-8F show a process of the present invention for autonomously verifying and dispensing a dose. FIG. 8A shows the doses arranged in an array within a secure terminal. FIGS. 8B-8C show an automated arm component gripping a dose and presenting it to a verification mechanism, in this case a camera. FIG. 8D shows the automated arm component dispensing the dose down a chute to be received. FIG. 8E shows the dose dispensed after traveling down the chute. FIG. 8F shows the chute in a rejection configuration such that the dose travels instead to a rejection reservoir.

FIG. 9A shows a gate assembly of the present invention in an open configuration. This would only occur after two-way biometric authentication and verification of the dose.

FIG. 9B shows the gate assembly of the present invention in a closed configuration. This would be the default state of the gate assembly prior to two-way biometric authentication and verification of the dose.

FIGS. 9C-9D show the gate assembly of the present invention with the cover removed.

FIG. 10A shows the interior of the terminal of the present invention with the gate closed and the dose order in the buffer.

FIG. 10B shows the interior of the terminal of the present invention with the gate open and the dose order in the buffer.

FIG. 10C shows the interior of the terminal of the present invention with the gate open and the dose order pushed out.

FIG. 10D shows the interior of the terminal of the present invention with the gate open and the dose order in the tray.

FIG. 10E shows the interior of the terminal of the present invention with the gate open and the dose order in the tray with the tray door opened.

FIG. 11 shows an overview of the remote dispensing terminal of the present invention.

FIG. 12 shows an overview of the quarantine chute of the remote dispensing terminal of the present invention for rejected/unverified doses.

### DETAILED DESCRIPTION OF THE INVENTION

Following is a list of elements corresponding to a particular element referred to herein:
100 supervisory computing platform
110 communication interface
120 processor
130 memory
140 authentication module
150 audit log database
160 session management module
200 remote dispensing unit
210 biometric sensing component
220 communication interface
230 processor
240 memory
250 user interface display
260 camera subsystem
270 secure preparation compartment
280 recipient interaction compartment
290 controlled transfer mechanism
295 locking mechanism
300 medication preparation module
310 liquid dispensing module
320 tablet dispensing module
330 labeling module
340 verification sensor
350 quarantine compartment
360 secure storage compartment
400 session camera
410 tray camera
420 overhead camera
500 external clinical information system
510 electronic health record system
520 network connection
1000 distributed system

In some aspects, the techniques described herein relate to a method for remotely supervising preparation and dispensing of medication to a recipient at a remote dispensing unit, the method including: (a) authenticating a recipient identity corresponding to the recipient using a first biometric sensing component; (b) authenticating, for one or more supervising entities, a supervising identity corresponding to the supervising entity using a second biometric sensing component; (c) establishing a real-time audiovisual communication session between the recipient at the remote dispensing unit and the one or more supervising entities; (d) receiving, at a supervisory computing platform, an electronic medication order and label data corresponding to the electronic medication order, originating from an external clinical information system; (e) authorizing one or more medication doses to be prepared at the remote dispensing unit based on the electronic medication order; (f) prior to release of a prepared dose, presenting, to the one or more supervising entities, verification data including (i) the label data, and (ii) at least one of pump feedback, tablet dispensing feedback, or captured image data of the prepared dose; and (g) actuating a controlled mechanical transfer mechanism of the remote dispensing unit to release the prepared dose to the recipient only after at least one of the one or more supervisory entities is authenticated and provides an affirmative release authorization.

In some aspects, the techniques described herein relate to a method, wherein both the recipient and at least one of the one or more supervisory entities are biometrically authenticated prior to actuation of the controlled mechanical transfer mechanism.

In some aspects, the techniques described herein relate to a method, further including enabling participation of a plurality of authenticated session participants in the real-time audiovisual communication session, wherein only participants assigned a supervisory dosing role are permitted to authorize actuation of the controlled mechanical transfer mechanism.

In some aspects, the techniques described herein relate to a method, further including, in response to a supervisory command, performing at least one of: cancelling preparing a dose, quarantining the prepared dose, locking a secure access door, unlocking the secure access door, or preventing transfer of the prepared dose to a recipient-accessible compartment.

In some aspects, the techniques described herein relate to a method, wherein the verification data further includes multiple camera perspectives including at least two of: a recipient view, a preparation-area view, and a transfer-mechanism view.

In some aspects, the techniques described herein relate to a method, wherein determining the identity of the recipient includes capturing biometric data using at least one of a fingerprint sensor, retina scanner, facial recognition component, or combination thereof.

In some aspects, the techniques described herein relate to a method, wherein the remote dispensing unit includes a private booth or enclosure configured for the real-time audiovisual communication session.

In some aspects, the techniques described herein relate to a method, wherein the medication includes a controlled substance.

In some aspects, the techniques described herein relate to a method, wherein the controlled substance includes methadone, buprenorphine, or both.

In some aspects, the techniques described herein relate to a method, wherein the one or more supervisory entities include pharmacists, nurses, or physicians. In some embodiments, the one or more supervisory entities may additionally include automated supervisory entities (e.g., machine learning models).

In some aspects, the techniques described herein relate to a method, further including evaluating recipient eligibility for releasing the prepared dose using an automated decision module configured to analyze recipient responses, biometric verification data, and historical session data, wherein releasing is conditionally authorized when predefined criteria are satisfied and wherein the one or more supervisory entities retain override authority.

In some aspects, the techniques described herein relate to a method, wherein the remote dispensing unit includes an internal isolation gate configured to prevent insertion of a recipient's hand into a secure preparation compartment during preparing or transferring the prepared dose.

In some aspects, the techniques described herein relate to a computing system for remotely supervising preparation and dispensing of medication to a recipient at a remote dispensing unit, the computing system including: (a) a communication component configured to communicatively couple to a plurality of remote dispensing units and to an external clinical information system; (b) a processor configured to execute computer-readable instructions; (c) a biometric authentication component configured to receive one or more recipient biometric identifiers from the recipient; and (d) a memory storing computer-readable instructions that, when executed by the processor, cause the computing system to: (i) receive the one or more recipient biometric identifiers associated with the recipient at the remote dispensing unit and confirm a recipient identity corresponding to the recipient based on the one or more biometric identifiers; (ii) receive, for one or more supervising entities, one or more supervisor biometric identifiers associated with the supervising entity and confirm a supervisor identity corresponding to the supervising entity based on the one or more supervisor biometric identifiers; (iii) establish a real-time audiovisual communication session between the recipient and at least one authenticated session participant; (iv) receive an electronic medication order and label data corresponding to the electronic medication order, originating from the external clinical information system; (v) transmit authorization signals to initiate one or more medication doses to be prepared at the remote dispensing unit based on the electronic medication order; (vi) display verification data to the one or more supervisory entities prior to dose release, the verification data including the label data and at least one of pump feedback, tablet dispensing feedback, or captured image data of a prepared dose; and (vii) transmit a control signal that actuates a controlled mechanical transfer mechanism at the remote dispensing unit to release the prepared dose to the recipient only after at least one of the one or more supervisory entities is authenticated and provides an affirmative release authorization.

In some aspects, the techniques described herein relate to a computing system, wherein the one or more supervisory entities authenticated using both a personal identification credential and a biometric identifier prior to transmission of the control signal that actuates the controlled mechanical transfer mechanism.

In some aspects, the techniques described herein relate to a computing system, wherein the computing system is configured to concurrently supervise and control a plurality of geographically distributed remote dispensing units.

In some aspects, the techniques described herein relate to a computing system, wherein the remote dispensing unit includes (i) a secure preparation compartment and (ii) a recipient interaction compartment separated by the controlled mechanical transfer mechanism.

In some aspects, the techniques described herein relate to a computing system, wherein the medication includes a controlled substance, and wherein the controlled substance includes methadone, buprenorphine, or both; or any other controlled or non-controlled medication.

In some aspects, the techniques described herein relate to a distributed computing system for remotely supervising preparation and dispensing of medication to a recipient at a remote dispensing unit, the distributed computing system including: (a) a plurality of remote dispensing units, each remote dispensing unit including: (i) a biometric sensing component configured to capture one or more recipient biometric identifiers of the recipient; (ii) a remote communication component; (iii) a remote processor; and (iv) a remote memory storing instructions that cause the remote dispensing unit to transmit the biometric data and verification data to a hub computing system; and (b) the hub computing system including: (i) a hub communication component configured to communicatively couple to the plurality of remote dispensing units and an external clinical information system; (ii) a hub processor; and (iii) a hub memory storing instructions that cause the hub computing system to: (A) confirm a recipient identity of the recipient based on the one or more recipient biometric identifiers; (B) confirm, for one or more supervising entities, a supervisor identity based on one or more supervisor biometric identifiers; (C) receive an electronic medication order and corresponding label data originating from the external clinical information system; (D) authorize one or more medication doses to be prepared at a selected remote dispensing unit based on the electronic medication order; (E) present verification data to the one or more supervisory entities prior to dose release; and (F) transmit a control signal to actuate a controlled mechanical transfer mechanism at the selected remote dispensing unit to release a prepared dose to the recipient only after at least one of the one or more supervisory entities is authenticated and provides an affirmative release authorization.

In some aspects, the techniques described herein relate to a distributed computing system, wherein the biometric sensing component includes at least one of a fingerprint sensor, retina scanner, facial recognition component, or combination thereof.

In some aspects, the techniques described herein relate to a distributed computing system, wherein the one or more supervisory entities include pharmacists, nurses, physicians, or a combination thereof.

In some embodiments, the recipient biometrics may comprise facial recognition. In these embodiments, the facial scan used for biometric authentication may be updated and overwritten in each scan instance to ensure that the most recent facial data for the recipient is maintained. In some embodiments, a dose may be flagged as “autoverified” by the system such that the dose is automatically dispensed upon positive biometric confirmation from the recipient and supervisor without requiring additional verification.

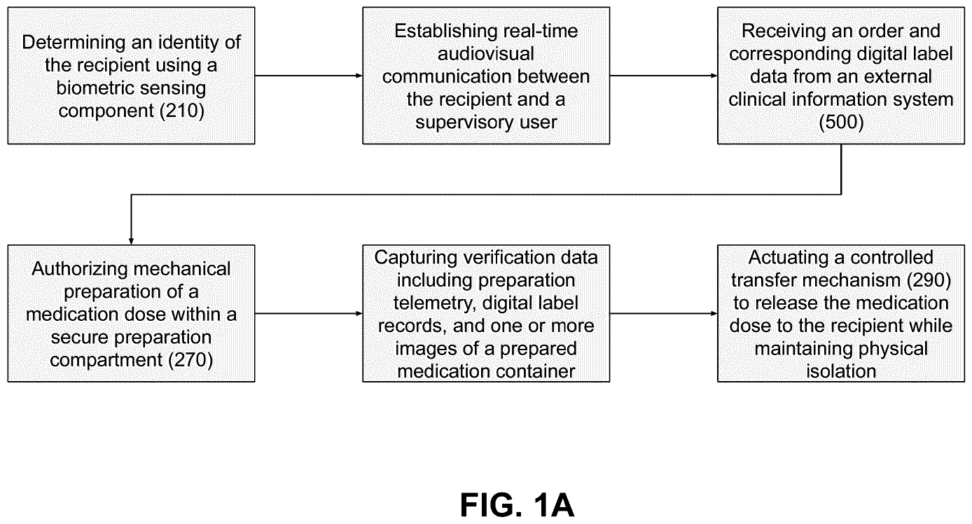

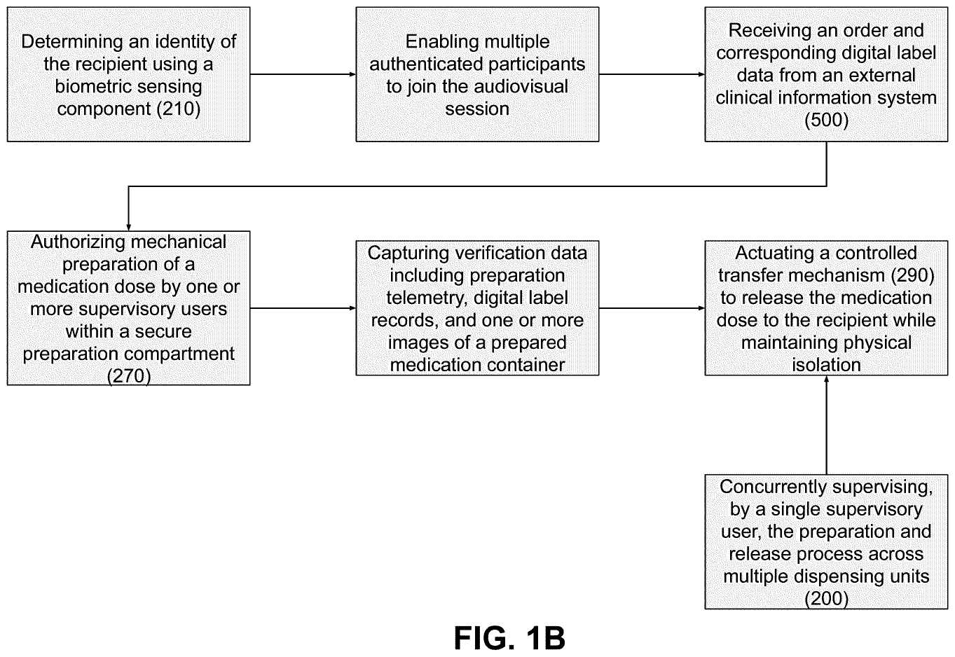

Referring now to FIG. 1A, a method is illustrated for remotely supervising preparation and controlled release of one or more medication doses to a recipient. The method may include determining an identity of the recipient using a biometric sensing component (210). The method may further include establishing real-time audiovisual communication between the recipient and a supervisory user; receiving an order and corresponding digital label data from an external clinical information system (500). The method may further include authorizing mechanical preparation of a medication dose within a secure preparation compartment (270). The method may further include capturing verification data including preparation telemetry, digital label records, and one or more images of a prepared medication container. The method may further include, upon supervisory authorization, actuating a controlled transfer mechanism (290) to release the medication dose to the recipient while maintaining physical isolation from the secure preparation compartment. The method may further include generating timestamped audit records and storing or transmitting such records to a secure data repository.

Referring now to FIG. 1B, an alternative method is illustrated in which multiple authenticated participants may join an audiovisual session associated with a dispensing event. Participants may include supervisory users, physicians, counselors, schedulers, or other authorized personnel. Only supervisory users possessing dosing authority may initiate or authorize preparation, quarantine, cancellation, locking, unlocking, or controlled release of medication doses. The method may further include concurrent supervision by a single supervisory user across a plurality of remote dispensing units (200), selective quarantine of prepared doses within a quarantine compartment (350), cancellation of preparation prior to release, and real-time locking or unlocking of a controlled transfer mechanism (290).

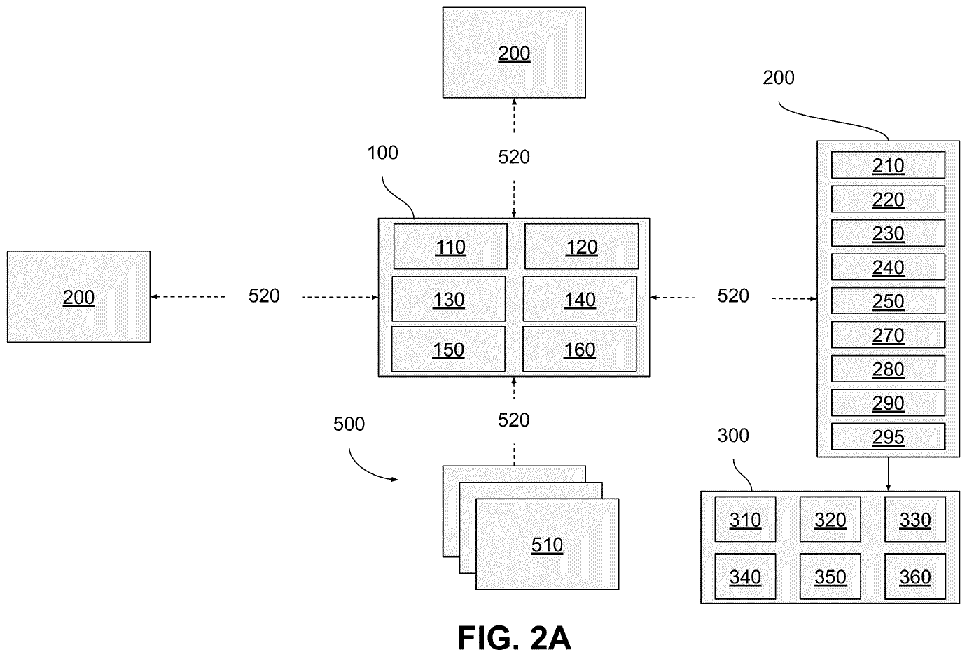

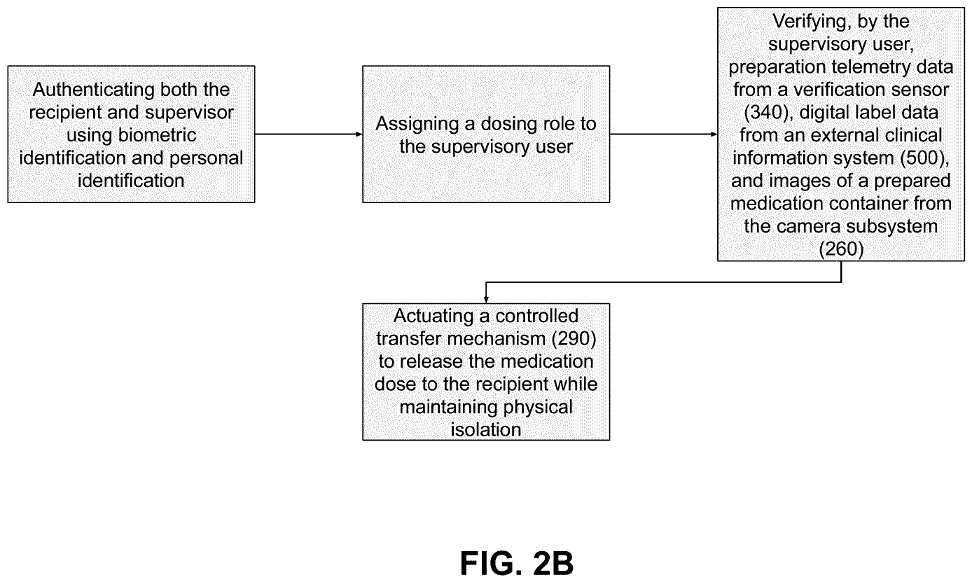

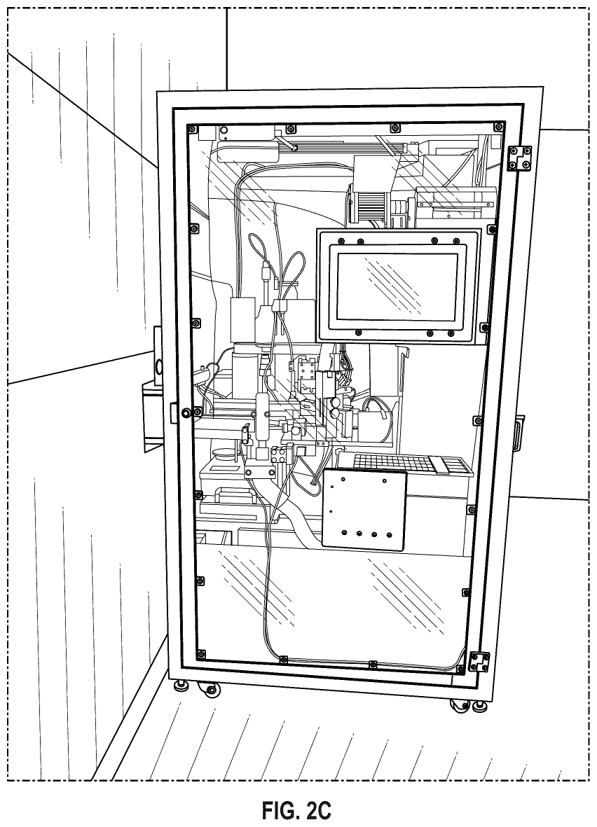

Referring now to FIG. 2A, a distributed system (1000) is illustrated for remotely supervised preparation and controlled dispensing of medication doses. The system may include a supervisory computing platform (100) communicatively coupled via a network connection (520) to a plurality of remote dispensing units (200) and one or more external clinical information systems (500), such as electronic health record systems (510). The supervisory computing platform (100) may include a communication interface (110), processor (120), memory (130), authentication module (140), audit log database (150), and session management module (160). The supervisory computing platform may authenticate supervisory users, maintain concurrent audiovisual sessions, receive medication orders and label data, present verification data, and transmit supervisory control signals to actuate preparation, quarantine, cancellation, locking, unlocking, or controlled release of medication doses at one or more remote dispensing units.

Each remote dispensing unit (200) may include a biometric sensing component (210), communication interface (220), processor (230), memory (240), user interface display (250), camera subsystem (260), secure preparation compartment (270), recipient interaction compartment (280), controlled transfer mechanism (290), and locking mechanism (295). The remote dispensing unit may further include a medication preparation module (300), which may comprise a liquid dispensing module (310), tablet dispensing module (320), labeling module (330), verification sensor (340), quarantine compartment (350), and secure storage compartment (360).

Referring now to FIG. 2B, a supervisory authentication and actuation flow is illustrated. In certain embodiments, both the recipient and the supervisory user are authenticated using biometric identification in addition to personal identification credentials prior to any mechanical actuation of medication release. Supervisory users may be assigned role-based permissions, and only users authenticated with a supervisory dosing role are permitted to transmit control signals that actuate preparation, quarantine, cancellation, locking, unlocking, or controlled release of medication doses. Prior to release, the supervisory user may verify a combination of preparation telemetry data from a verification sensor (340), digital label data received from an external clinical information system (500), and one or more captured images of a prepared medication container obtained from the camera subsystem (260).

FIG. 2C illustrates an example remote dispensing unit (200) with a transparent enclosure revealing internal robotic automation components including a medication preparation module (300), liquid dispensing module (310), labeling module (330), verification sensors (340), and internal transfer pathways. The unit includes a secure preparation compartment (270) physically isolated from a recipient interaction compartment and configured to mechanically prepare and verify medication doses prior to controlled release.

The user interface may display a centralized queue of recipients across multiple remote dispensing units, one or more active audiovisual session windows, and role-based control elements including approve, cancel, quarantine, lock, and unlock controls. The interface may further display recipient identity data, timestamps, and session status indicators. The supervisory user interface may be generated by the supervisory computing platform (100) and presented on a supervisory terminal or computing device.

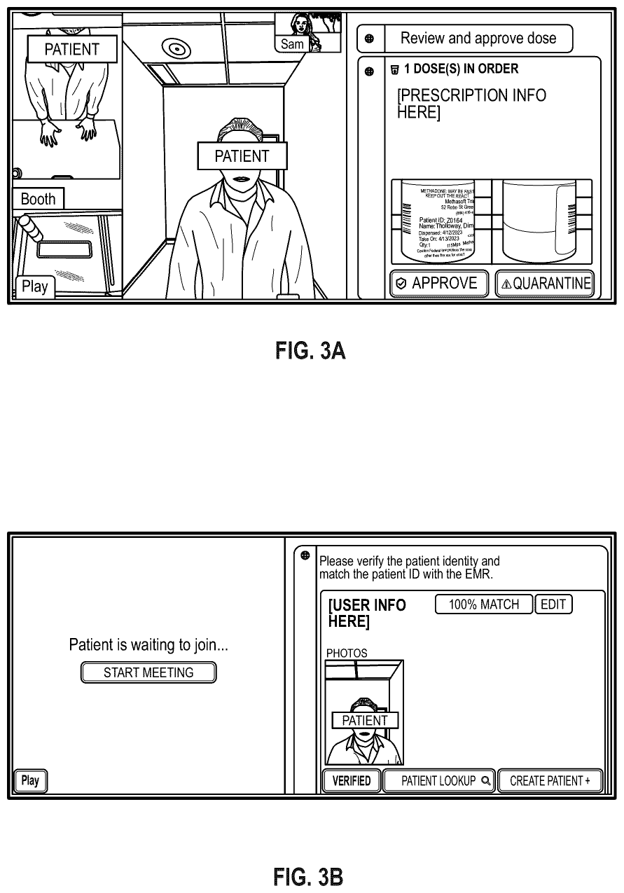

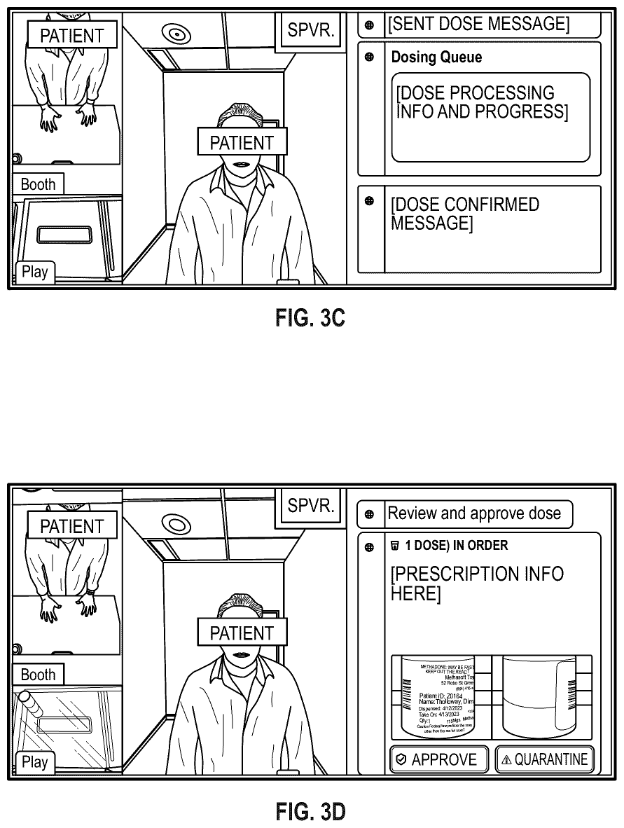

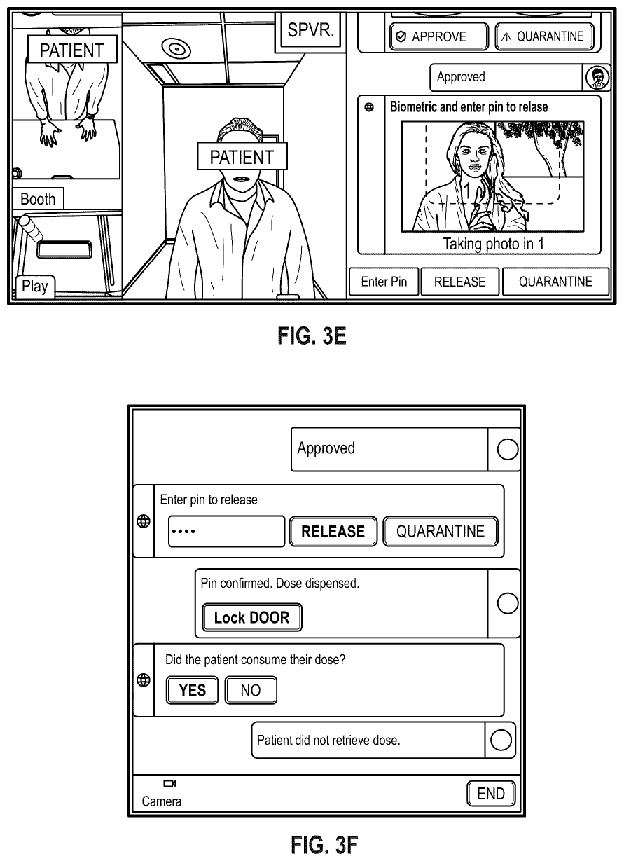

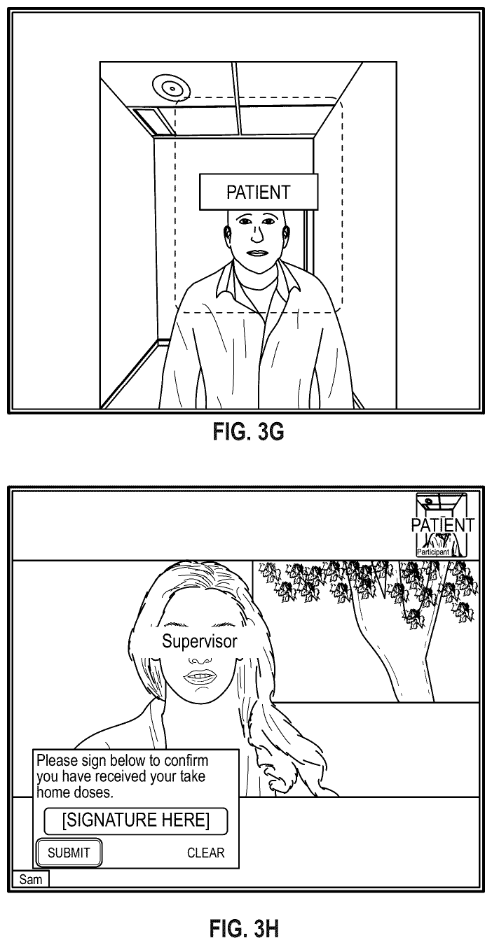

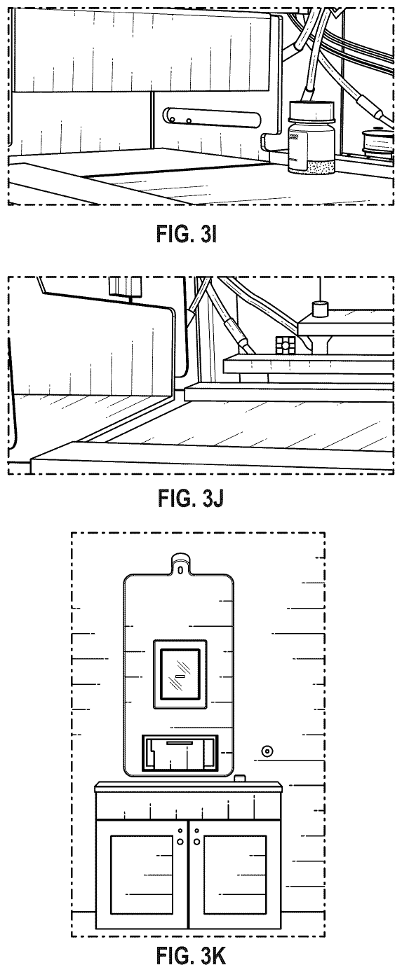

Referring now to FIG. 3A, an example supervisory user interface is illustrated presenting verification data associated with a medication preparation event. The interface may display digital label records, preparation telemetry values, one or more captured images of a prepared medication container, and simultaneous camera perspectives including a recipient view (400), tray view (410), and overhead view (420). Release controls may require entry of a personal identification credential and biometric confirmation prior to actuation of a controlled transfer mechanism (290). The interface may further provide access to historical audit records stored within the audit log database (150).

Referring now to FIG. 3B, an example supervisory user interface is illustrated presenting recipient identity verification data prior to initiation of a live audiovisual session. The interface may display a biometric comparison result, a stored recipient photograph, and associated identifying information including name, identification number, and date of birth. A supervisory user may confirm identity prior to commencing real-time communication and may subsequently access a corresponding electronic medical record through a separate clinical information system. The interface may further include controls for verification confirmation, patient lookup, or creation of a new recipient profile.

Referring now to FIG. 3C, an example supervisory user interface is illustrated presenting receipt of a medication order originating from an external clinical information system and a corresponding preparation queue. The interface may display one or more active audiovisual views of a recipient, including a frontal recipient view, an overhead verification view, and a preparation or tray view. A supervisory user may review order information, preparation status indicators, and queue progress associated with one or more recipients. The interface may further provide selectable controls enabling the supervisory user to authorize preparation of a medication dose, pause processing, or cancel an order prior to mechanical preparation. In certain embodiments, preparation is not initiated until explicit supervisory authorization is received, and all actions may be timestamped and stored within an audit record.

Referring now to FIG. 3D, an example supervisory user interface is illustrated presenting verification data associated with a prepared medication dose prior to mechanical release to a recipient. The interface may display preparation telemetry values including a measured or dispensed dose amount, one or more digital label records received from an external clinical information system (500), and multiple captured images of a prepared medication container obtained from a camera subsystem. Simultaneous audiovisual views of the recipient and preparation environment may also be presented. The supervisory user may be provided selectable controls to approve release of the medication dose or to quarantine the prepared dose. In certain embodiments, approval of release requires entry of a personal identification credential and biometric authentication prior to actuation of a controlled transfer mechanism. Until such supervisory authorization is completed, the prepared medication dose remains physically isolated from the recipient.

Referring now to FIG. 3E-F, an example supervisory authentication interface is illustrated in which a supervisory user is required to complete dual-factor identity verification prior to actuation of a controlled transfer mechanism. The interface may present a live biometric capture window configured to obtain facial or other biometric data from the supervisory user and a personal identification credential entry field. Mechanical release controls remain disabled until both biometric authentication and credential validation are successfully completed. In certain embodiments, failure to satisfy either authentication requirement results in continued physical isolation of the prepared medication dose and may trigger audit logging, lockout intervals, or supervisory alerts. The dual-authentication sequence provides role-segmented authority enforcement and prevents unauthorized mechanical release of medication doses.

Referring now to FIG. 3G, an example recipient biometric re-authentication interface is illustrated in which a recipient is required to complete biometric identity confirmation immediately prior to mechanical actuation of a controlled transfer mechanism. The interface may present a live capture window configured to obtain facial or other biometric data from the recipient and may include a timed capture or countdown indicator to guide positioning. In certain embodiments, the recipient biometric verification occurs after supervisory approval and supervisory dual-factor authentication have been completed but before physical release of the prepared medication dose. Mechanical release controls remain disabled until recipient identity confirmation is successfully validated, thereby ensuring that the prepared medication dose is dispensed directly to the verified recipient and not to an intermediary. The sequence of supervisory authentication followed by recipient re-authentication provides layered identity assurance, diversion prevention, and audit traceability.

Referring now to FIG. 3H, an example recipient acknowledgment interface is illustrated in which the recipient provides an electronic confirmation of receipt of one or more dispensed medication doses. The interface may be presented within an audiovisual session window and may include a digital signature capture field, confirmation prompts, and submission controls. In certain embodiments, the acknowledgment occurs after mechanical release of an observed dose and/or one or more take-home doses and is associated with timestamped audit records stored within an audit log database. The acknowledgment may be linked to recipient identity verification data, supervisory approval records, and dose preparation telemetry to establish a verifiable chain-of-custody. The electronic signature may be stored as a digital artifact and may be transmitted to an external clinical information system or retained within a supervisory computing platform for compliance, documentation, and dispute resolution purposes.

Referring now to FIGS. 3I-J, an internal isolation gate or barrier is illustrated within the remote dispensing unit separating a secure preparation compartment from a recipient interaction compartment. The isolation gate may comprise a sliding panel, hinged door, or vertically actuated barrier configured to remain closed during mechanical preparation and only permit transfer of a prepared medication container into a controlled transfer mechanism. In certain embodiments, the isolation gate prevents insertion of a recipient's hand or foreign object into the preparation area and thereby maintains physical separation between robotic dispensing components and the recipient environment. Sensors, actuators, or position detectors associated with the isolation gate may generate audit records corresponding to open and closed states and transfer events.

Referring now to FIG. 3K, the present invention features a front-of-clinic dispensing station designed for supervised remote medication encounters. The unit includes a patient-facing touchscreen enabling real-time audio-visual telehealth communication with a licensed clinician, identity confirmation, and session guidance; an overhead, bird's-eye camera providing continuous visual oversight of the dispensing area; and a secure, closed medication compartment engineered to limit access and support chain-of-custody controls. During a session, the clinician can observe the patient and the dispensing surface simultaneously, verify patient identity, authorize dose preparation, and document the encounter within the connected clinical software. The physical layout supports single-patient use, privacy positioning, and clear sightlines for both patient interaction and safety monitoring. Integrated hardware and software controls enable time-stamped session logs, audit trails, and remote lock/unlock functionality, while enclosure design, camera placement, and tamper-resistant features contribute to diversion-prevention, error reduction, and transparent dose preparation. This figure illustrates the patient interaction point and supervisory safeguards that complement the digital queue and clinical workflow shown in preceding subfigures.

A recipient is required to complete biometric identity confirmation immediately prior to mechanical actuation of a controlled transfer mechanism. The interface may present a live capture window configured to obtain facial or other biometric data from the recipient and may include a timed capture or countdown indicator to guide positioning. In certain embodiments, the recipient biometric verification occurs after supervisory approval and supervisory dual-factor authentication have been completed but before physical release of the prepared medication dose. Mechanical release controls remain disabled until recipient identity confirmation is successfully validated, thereby ensuring that the prepared medication dose is dispensed directly to the verified recipient and not to an intermediary. The sequence of supervisory authentication followed by recipient re-authentication provides layered identity assurance, diversion prevention, and audit traceability.

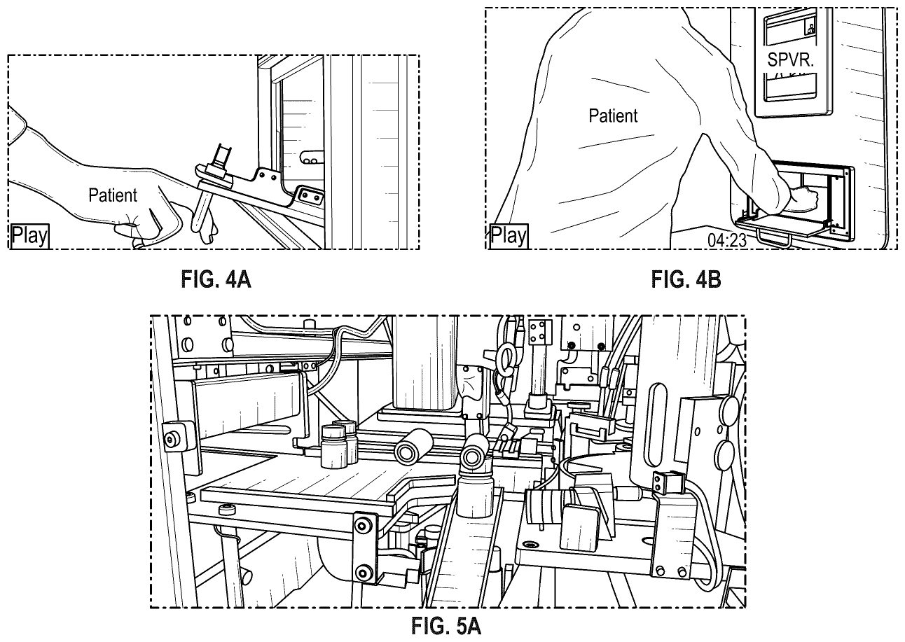

Referring now to FIGS. 4A-4B, an example controlled transfer mechanism is illustrated in an unlocked state following supervisory authorization and dual-factor authentication. The controlled transfer mechanism may comprise a selectively lockable door, drawer, or pass-through structure positioned between a secure preparation compartment and a recipient interaction compartment. In certain embodiments, the mechanism remains mechanically locked until receipt of authenticated supervisory control signals and recipient identity verification. Upon release, the recipient may manually access the prepared medication dose while the secure preparation compartment remains physically isolated. The controlled transfer mechanism may include sensors, status indicators, or locking actuators configured to generate timestamped audit records associated with each unlock and retrieval event. As depicted in FIG. 4B, the controlled transfer door is then unlocked for recipient retrieval of medication. The controlled transfer mechanism is actuated and the recipient retrieves the dose following supervisory authorization.

Referring now to FIGS. 5A-5C, an example automated quarantine mechanism is illustrated in association with an internal robotic handling assembly configured to selectively remove prepared medication containers from an intermediate buffer platform. The robotic assembly may comprise a multi-axis actuator, gripper, or end-effector mechanism operable to engage sealed medication containers positioned within a staging or verification buffer. In certain embodiments, the robotic assembly retrieves the medication container following completion of identity verification, sensor confirmation, or supervisory control signals indicating quarantine or diversion from standard dispensing flow.

The retrieved medication container may be transferred along an internal guide track, conveyor, or positioning rail while optical sensors, weight sensors, or positional detection components perform container identification, status validation, or audit confirmation. In some embodiments, the system may generate timestamped audit records associated with each robotic retrieval, transfer action, or quarantine designation event.

Upon determination that the medication container is to be quarantined, the robotic assembly may position the container above a secured quarantine chute, conduit, or enclosed transfer tube configured to direct the container into a restricted internal medication compartment inaccessible to external users. The quarantine chute may comprise transparent or opaque materials and may include anti-reverse features, tamper-resistant geometry, or sensor-based confirmation of container passage.

The restricted internal medication compartment may remain physically isolated from recipient-accessible compartments and may be configured for subsequent pharmacist or authorized personnel access only. In certain embodiments, the quarantine mechanism may be triggered automatically based on verification discrepancies, expiration parameters, dosage irregularities, supervisory input, or system fault detection. The described configuration enables automated segregation of medication containers while maintaining continuous auditability, physical security, and controlled handling within the automated dispensing apparatus.

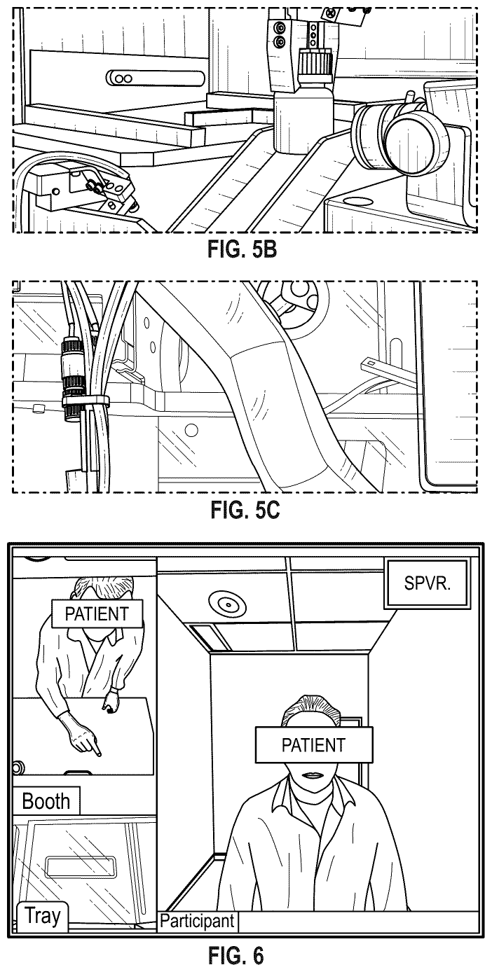

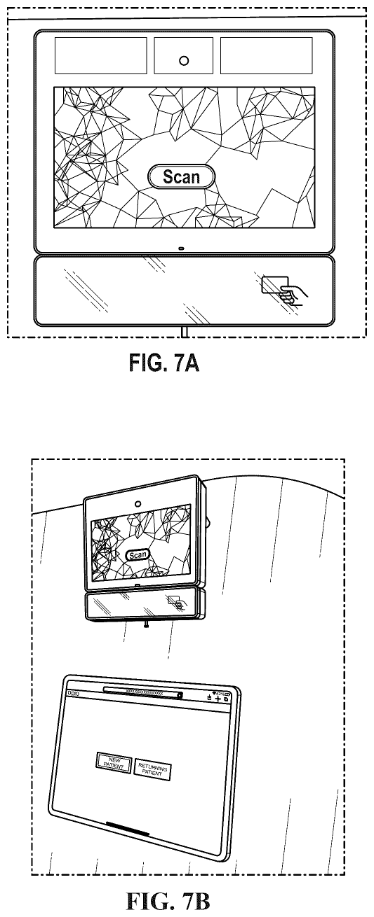

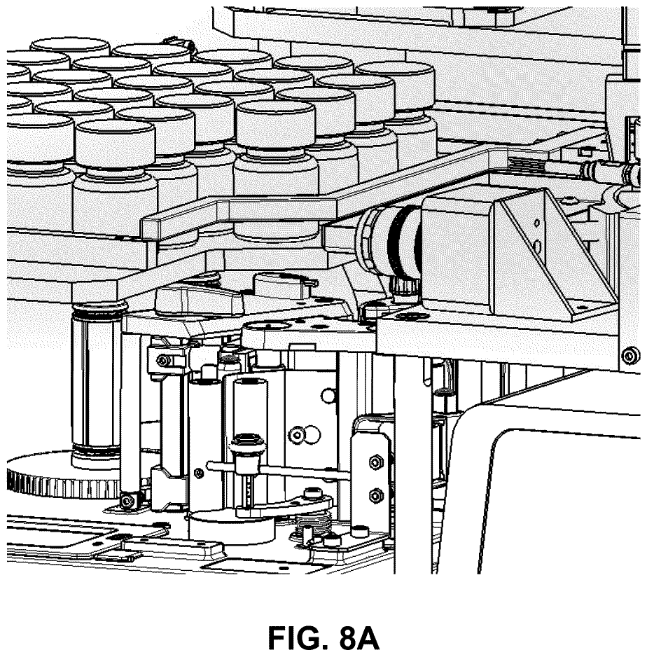

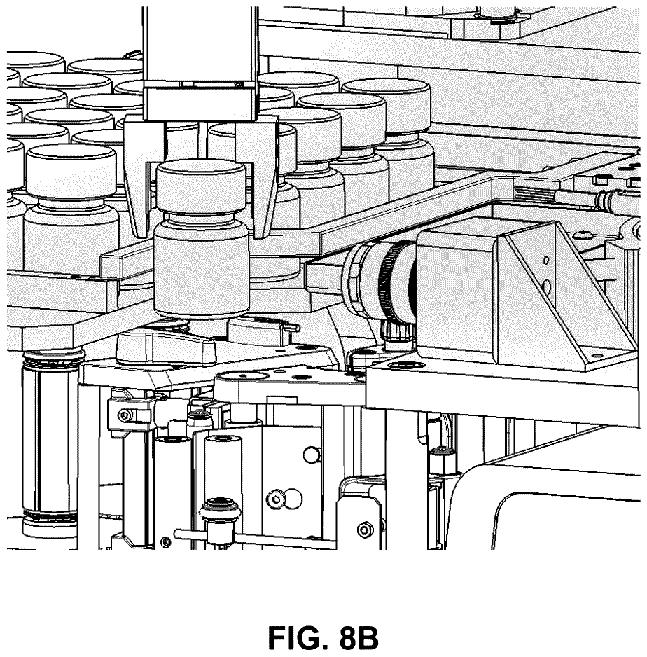

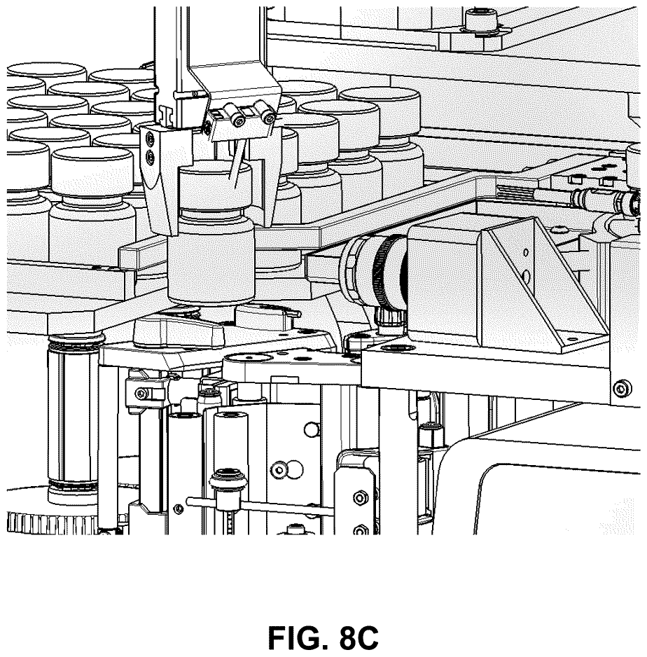

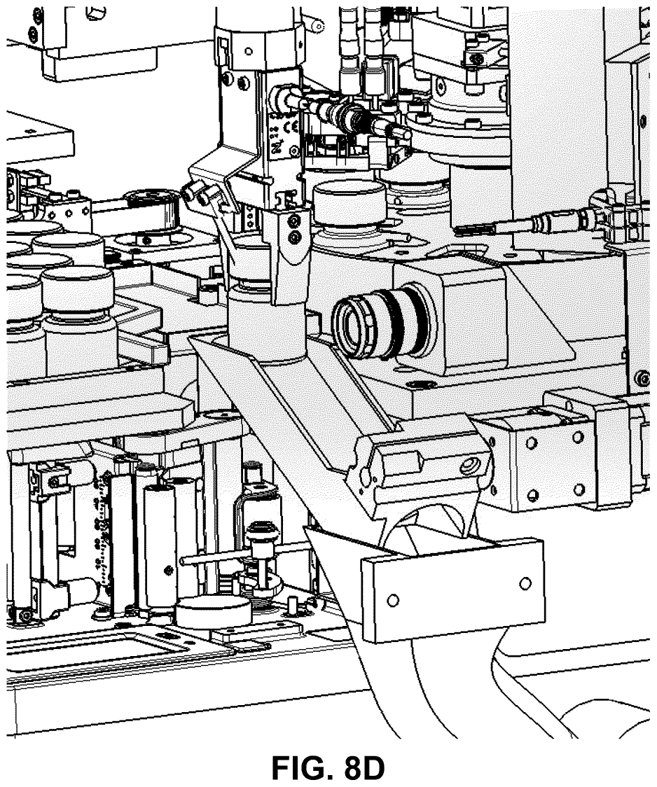

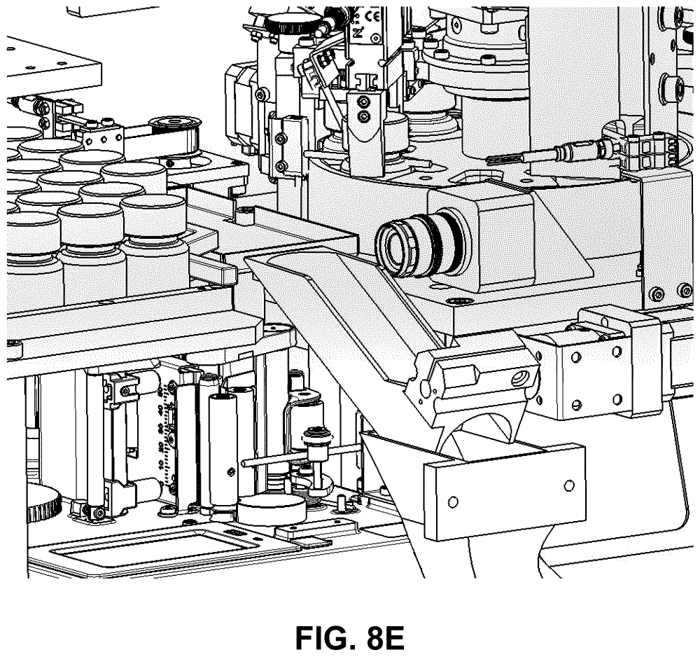

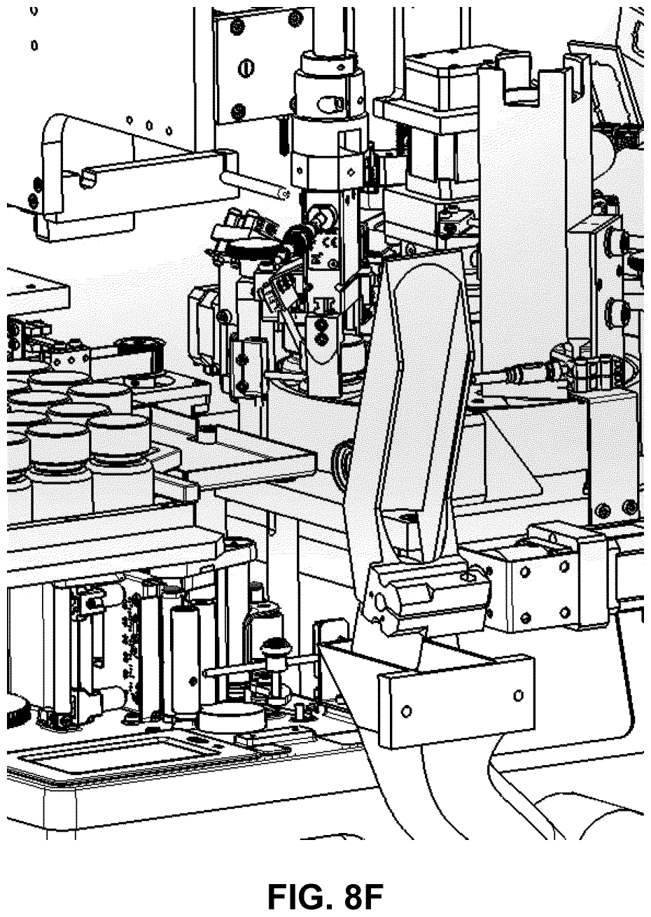

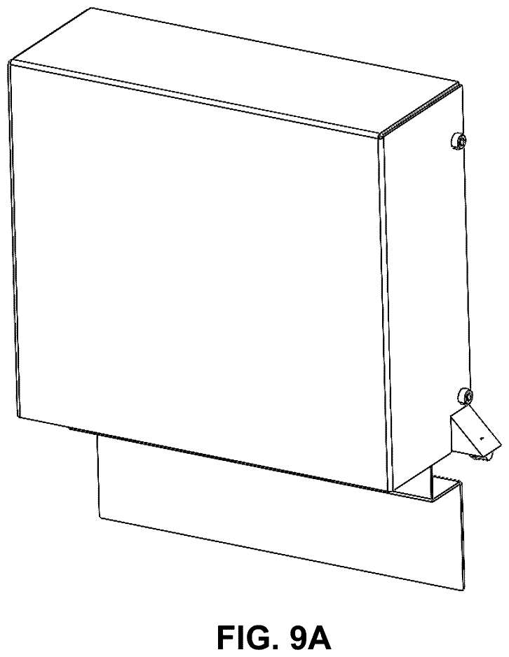

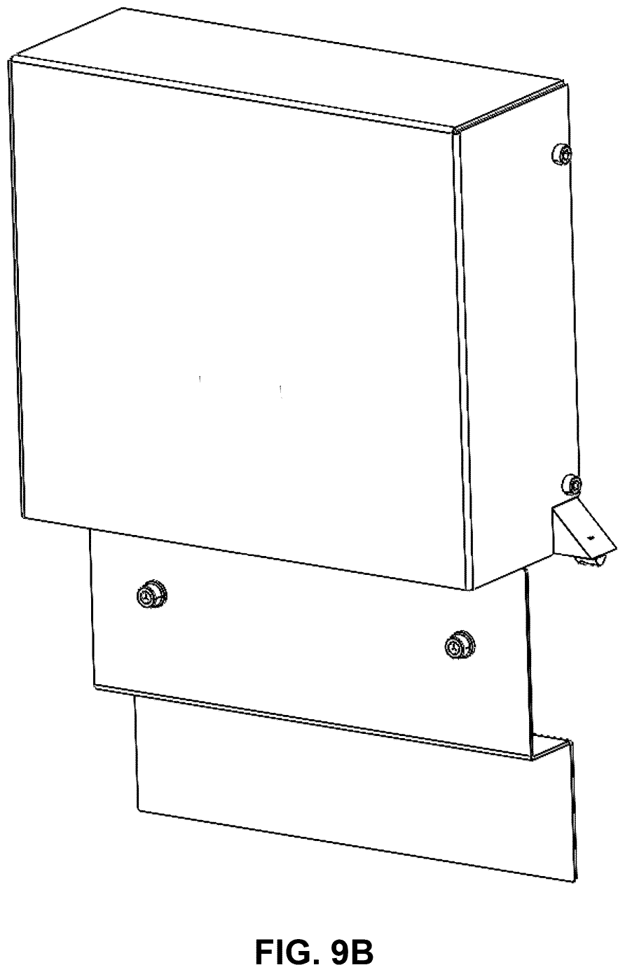

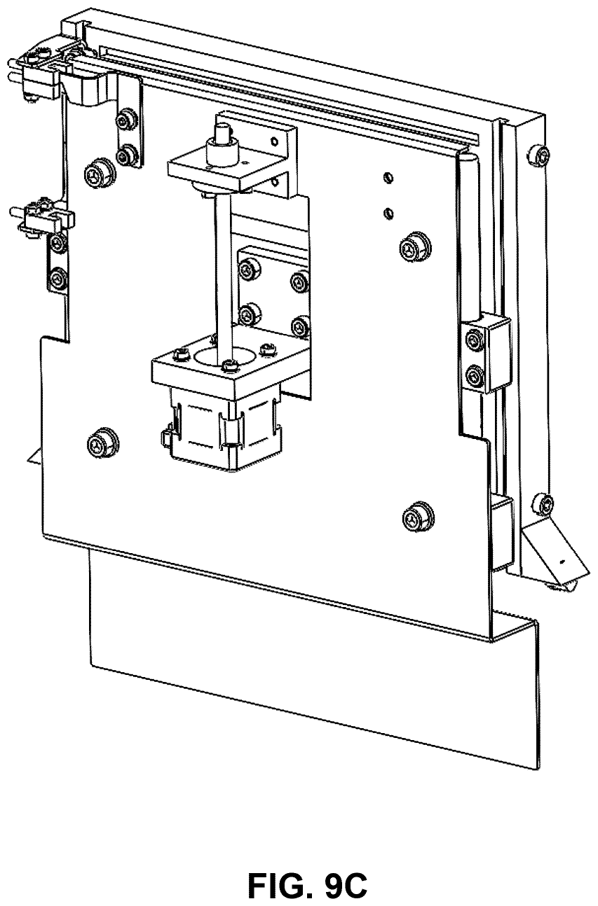

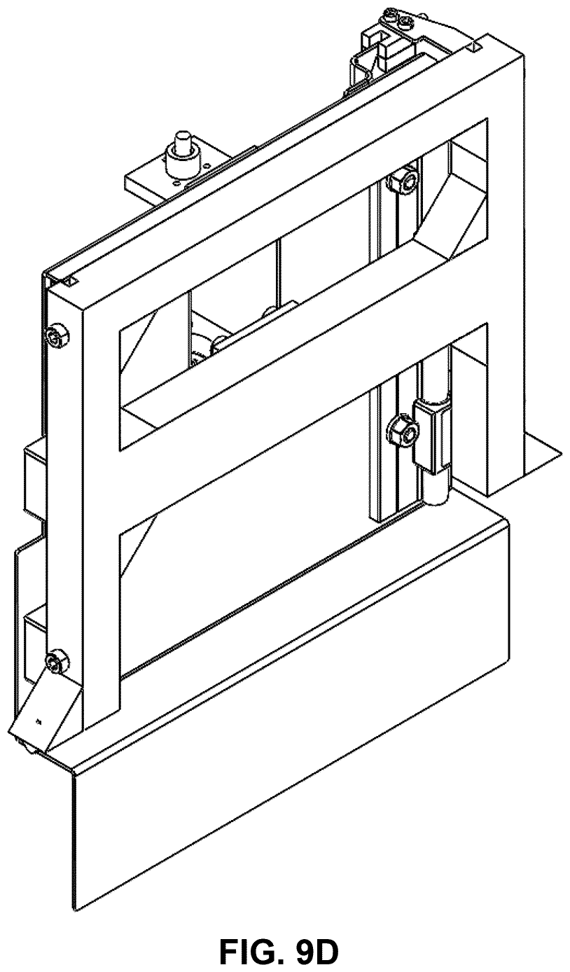

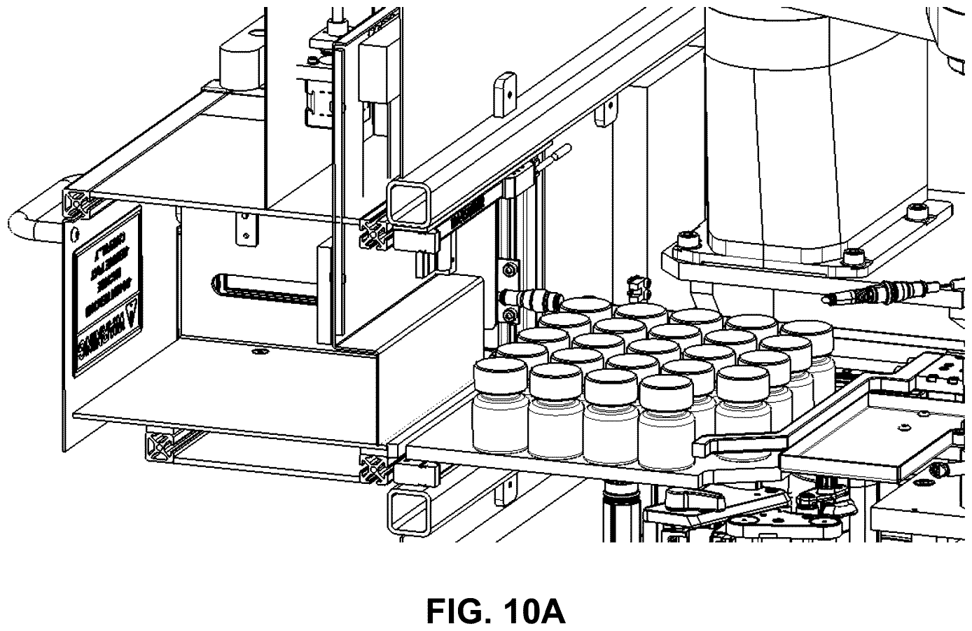

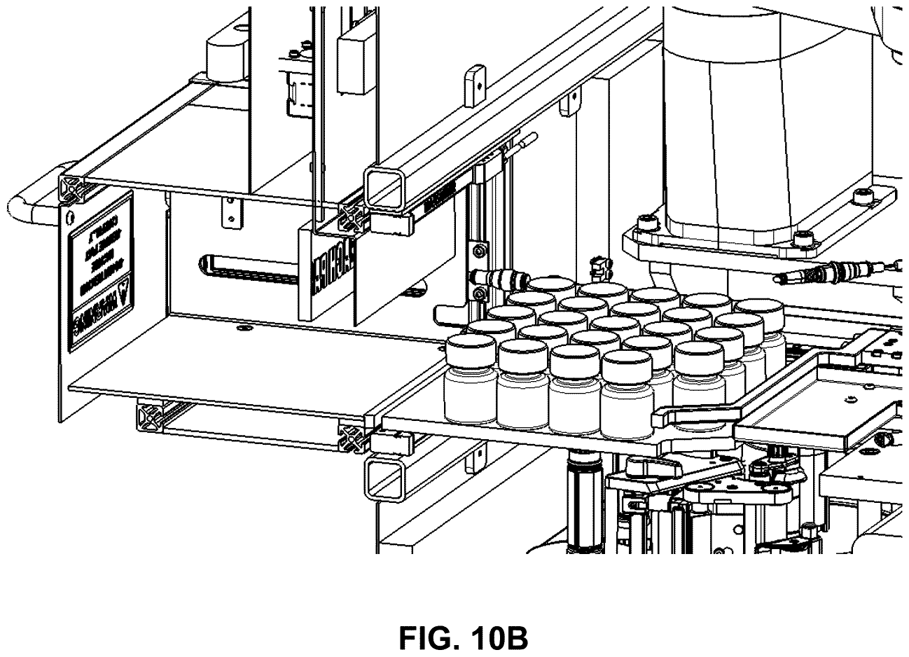

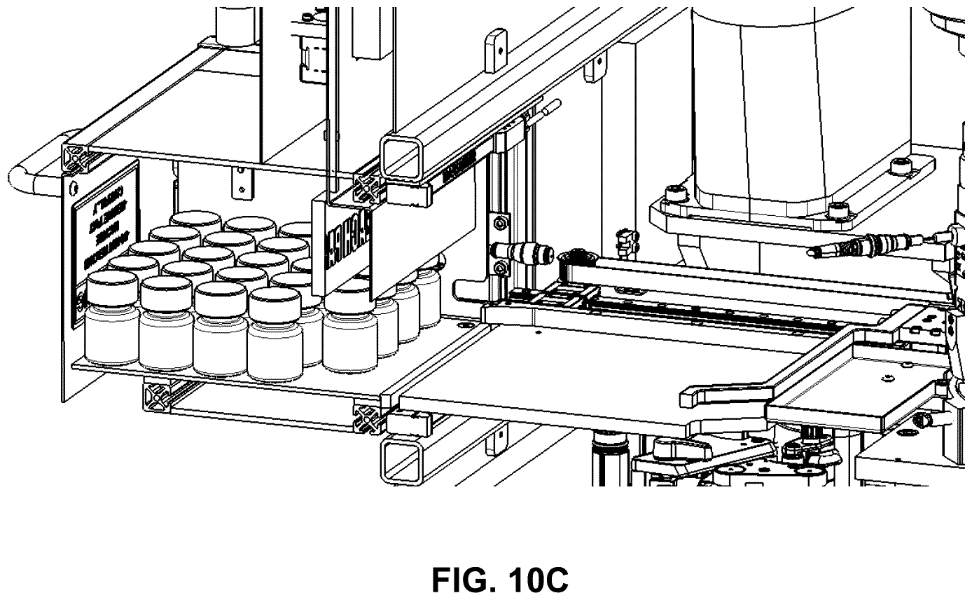

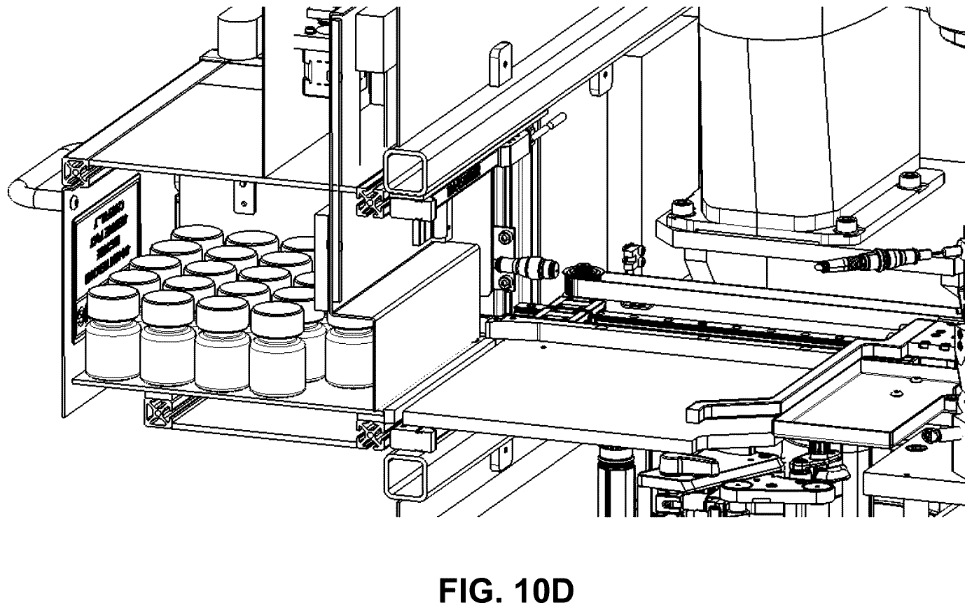

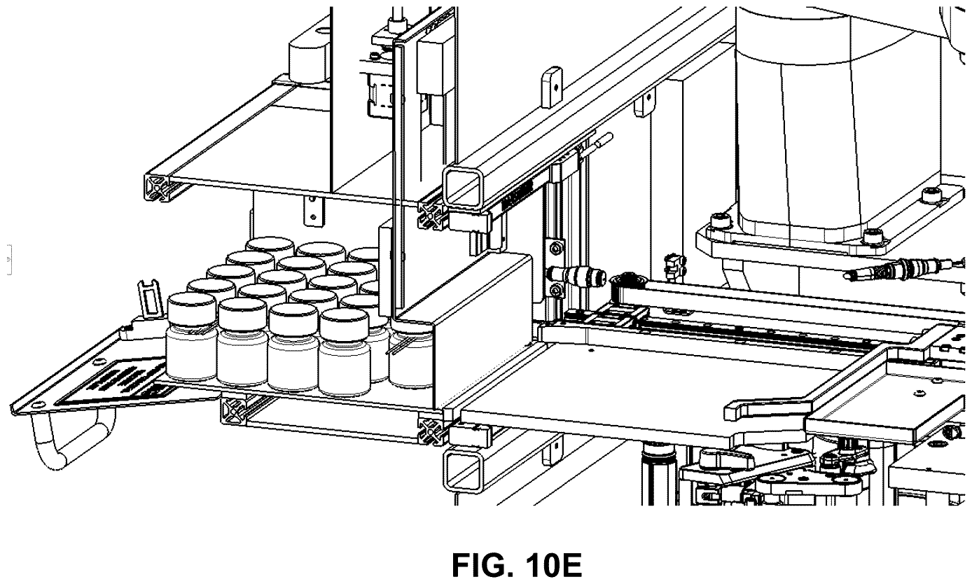

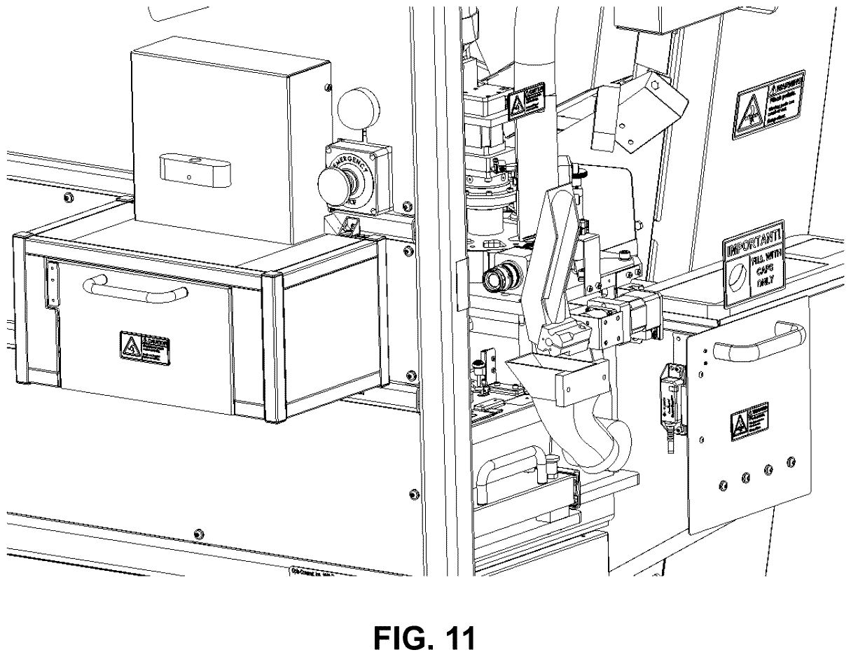

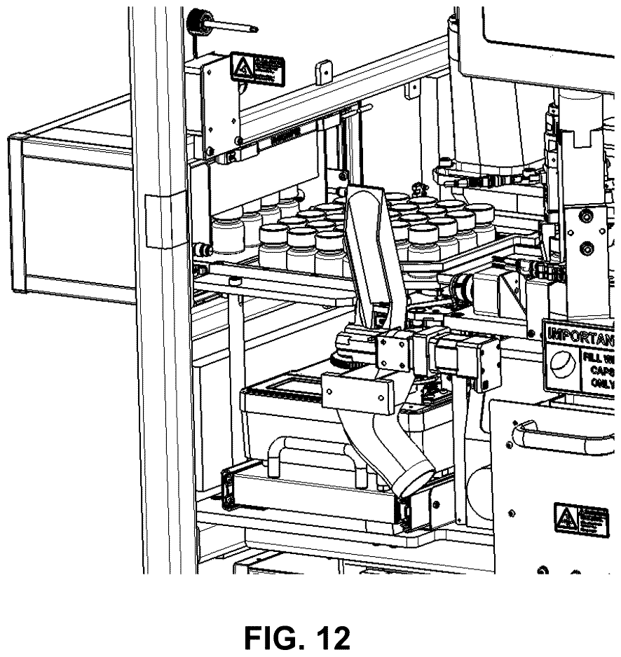

Referring now to FIG. 6, an example supervisory monitoring interface is illustrated presenting multiple simultaneous camera views accessible to authenticated clinical personnel. The interface may comprise a first visual feed directed toward a documentation or signing surface, a second visual feed directed toward an internal robotic preparation or dispensing compartment, and a third visual feed directed toward a recipient interaction booth or retrieval tray. In certain embodiments, the system is configured to display the feeds concurrently within a unified graphical interface to enable real-time visual confirmation of patient presence, medication preparation status, and controlled transfer events. Access to the monitoring interface may be restricted to authorized supervisory users through one or more authentication mechanisms. The system may further generate synchronized timestamped video records, event logs, and audit data associated with each dispensing session to support regulatory compliance, chain-of-custody verification, and operational accountability.

Medication doses may include liquid, tablet, capsule, packet, blister, or other dosage forms. The controlled transfer mechanism (290) may include a sliding drawer, dual-door pass-through, gated chute, rotating carousel, vertical lift, or other selectively lockable transfer structure. Communication between components may occur over wired or wireless networks and may include buffered or store-and-forward modes during transient connectivity interruption. A single supervisory user may concurrently supervise and control medication preparation and dispensing across a plurality of geographically distributed remote dispensing units without distance limitation while maintaining independent verification streams and audit logs for each session.

In certain embodiments, an automated decision module or machine-learning model may be utilized to assist or conditionally authorize preparation or release of medication doses based on predefined criteria. Such criteria may include recipient responses to structured questions, historical compliance data, biometric consistency, impairment assessment indicators, or confidence scores generated from verification inputs. The automated decision module may operate in a decision-support capacity or may conditionally authorize release when threshold criteria are satisfied, while maintaining the ability for a supervisory user to review, override, or revoke automated determinations. The automated decision module may be configured according to jurisdictional policies, clinical protocols, or risk-based thresholds and may generate an audit record of all automated and supervisory decisions.

### EXAMPLE

The following is a non-limiting example of the present invention. It is to be understood that this example is illustrative only and is not intended to limit the present invention in any way. Equivalents, variations, and substitutions are within the scope of the present invention.

In one illustrative workflow, a supervisory clinician remotely supervises medication preparation and dispensing for recipients located at multiple geographically distributed sites throughout a period of time. Recipients from multiple locations are organized into a centralized queue presented on a supervisory interface. Only recipients requiring attention or engaged in an active session may be displayed, while completed sessions remain retrievable in historical records. Each recipient may be associated with a display identifier shown alongside name and location information. The centralized queue may support multiple concurrent audiovisual sessions, allowing the supervisory clinician to maintain visibility of more than one active recipient session simultaneously and to exit and return to sessions as needed. Session initiation and completion actions may be explicitly confirmed and automatically timestamped.

During an interaction between a supervisory clinician and a recipient, identity information such as name, date of birth, and consent status may be collected. For new recipients, biometric identity data may be established using facial recognition or other biometric methods. For returning recipients, identity confirmation may occur through biometric comparison with stored records. Based on verified identity, the supervisory clinician may access the recipient's profile, including responses to clinical or administrative questions. Medication preparation may be initiated in response to an order transmitted from an external clinical information system associated with the recipient's location.

Medication doses may be prepared automatically within a secure preparation compartment and may require supervisory authorization before release. Individual observed doses may be processed immediately, while additional doses may enter a paused or quarantined state pending supervisory review. For grouped doses, one or more images of prepared containers may be presented for visual verification prior to release. Prepared doses may remain physically isolated from a recipient-accessible transfer area until the supervisory clinician authorizes mechanical actuation of a controlled transfer mechanism.

The supervisory clinician may observe the recipient ingesting or otherwise receiving medication through real-time audiovisual communication. The clinician may record whether prepared doses were successfully retrieved, and if not, may signal that doses should be quarantined or secured. While automated preparation processes occur at one site, the supervisory clinician may join additional recipient sessions at other sites while maintaining monitoring capability. All preparation, verification, release, quarantine, and communication events may be timestamped and stored in an audit record.

On the recipient side, new recipients may provide identifying information and establish biometric identity data for future verification. Returning recipients may confirm identity using previously stored biometric credentials. Recipients may respond to required clinic or facility questions while awaiting supervisory interaction and may communicate with the supervisory clinician via audiovisual session. Upon authorization, recipients may retrieve prepared doses from a controlled transfer area and may be instructed to ingest or secure medication as appropriate. All recipient interactions may be logged with associated timestamps.

In a first illustrative scenario, a returning recipient enters a remote dispensing enclosure. The enclosure may prompt for identifying information or biometric confirmation. If biometric confirmation fails or identification credentials are unavailable, the enclosure may capture one or more images for identity verification by the supervisory clinician. Once identity is confirmed, the supervisory session begins. Recipient identifying information may remain visible on the supervisory interface throughout the session, and authentication scores or image comparisons may be presented if needed. The supervisory clinician may update or correct recipient profiles and may capture additional images where clarity is insufficient.

In a second illustrative scenario, a new recipient enters a remote dispensing enclosure and provides identifying information, agrees to terms of use, and optionally submits identification documentation. Upon completion of onboarding, a supervisory session begins and captured documentation may be stored in the recipient profile. The supervisory clinician may verify information, check for duplicate accounts, and assign a unique recipient identifier linked to an external clinical information system.

In these and other scenarios, medication orders may be transmitted from an external clinical information system to the dispensing system, and preparation telemetry, digital label data, and captured images of prepared medication containers may be presented to the supervisory clinician prior to release. Authorization may require entry of a personal identification credential and biometric confirmation. The supervisory clinician may confirm ingestion of an observed dose, authorize or cancel preparation of additional doses, quarantine prepared doses, or secure doses pending retrieval. If doses are not retrieved, the system may lock controlled transfer mechanisms and notify authorized onsite personnel. A supervisory clinician may manage multiple concurrent recipient sessions across multiple locations while automated preparation occurs independently at each dispensing unit.

The computer system can include a desktop computer, a workstation computer, a laptop computer, a netbook computer, a tablet, a handheld computer (including a smartphone), a server, a supercomputer, a wearable computer (including a SmartWatch™), or the like and can include digital electronic circuitry, firmware, hardware, memory, a computer storage medium, a computer program, a processor (including a programmed processor), an imaging apparatus, wired/wireless communication components, or the like. The computing system may include a desktop computer with a screen, a tower, and components to connect the two. The tower can store digital images, numerical data, text data, or any other kind of data in binary form, hexadecimal form, octal form, or any other data format in the memory component. The data/images can also be stored in a server communicatively coupled to the computer system. The images can also be divided into a matrix of pixels, known as a bitmap that indicates a color for each pixel along the horizontal axis and the vertical axis. The pixels can include a digital value of one or more bits, defined by the bit depth. Each pixel may comprise three values, each value corresponding to a major color component (red, green, and blue). A size of each pixel in data can range from 8 bits to 24 bits. The network or a direct connection interconnects the imaging apparatus and the computer system.

The term “processor” encompasses all kinds of apparatus, devices, and machines for processing data, including by way of example a programmable microprocessor, a microcontroller comprising a microprocessor and a memory component, an embedded processor, a digital signal processor, a media processor, a computer, a system on a chip, or multiple ones, or combinations, of the foregoing. The apparatus can include special-purpose logic circuitry, e.g., an FPGA (field programmable gate array) or an ASIC (application-specific integrated circuit). Logic circuitry may comprise multiplexers, registers, arithmetic logic units (ALUs), computer memory, look-up tables, flip-flops (FF), wires, input blocks, output blocks, read-only memory, randomly accessible memory, electronically-erasable programmable read-only memory, flash memory, discrete gate or transistor logic, discrete hardware components, or any combination thereof. The apparatus also can include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures. The processor may include one or more processors of any type, such as central processing units (CPUs), graphics processing units (GPUs), special-purpose signal or image processors, field-programmable gate arrays (FPGAs), tensor processing units (TPUs), and so forth.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, subprograms, or portions of code). A computer program can be deployed to be executed on one computer or on multiple computers that are located at one site or distributed across multiple sites and interconnected by a communication network.

Embodiments of the subject matter and the operations described herein can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, a data processing apparatus.

A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or can be included in, one or more separate physical components or media (e.g., multiple CDs, drives, or other storage devices). The operations described in this specification can be implemented as operations performed by a data processing apparatus on data stored on one or more computer-readable storage devices or received from other sources.

Program code embodied on a computer readable medium may be transmitted using any appropriate medium, including but not limited to wireless, wireline, optical fiber cable, R. F, Bluetooth, storage media, computer buses, etc., or any suitable combination of the foregoing. Computer program code for carrying out operations for aspects of the present disclosure may be written in any combination of one or more programming languages, including an object oriented programming language such as Java, Smalltalk, C #, Ruby, or the like, conventional procedural programming languages, such as Pascal, FORTRAN, BASIC, or similar programming languages, programming languages that have both object-oriented and procedural aspects, such as the “C” programming language, C++, Python, or the like, conventional functional programming languages such as Scheme, Common Lisp, Elixir, or the like, conventional scripting programming languages such as PHP, Perl, Javascript, or the like, or conventional logic programming languages such as PROLOG, ASAP, Datalog, or the like.

The program code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application-specific integrated circuit).

Processors suitable for the execution of a computer program include, by way of example, both general and special purpose microprocessors, and any one or more processors of any kind of digital computer. Generally, a processor will receive instructions and data from a read-only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto-optical disks, or optical disks.

However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non-volatile memory, media and memory devices, including by way of example semiconductor memory devices, e.g., EPROM, EEPROM, and flash memory devices; magnetic disks, e.g., internal hard disks or removable disks; magneto-optical disks; and CD-ROM and DVD-ROM disks. The processor and the memory can be supplemented by, or incorporated in, special purpose logic circuitry.

Computers typically include known components, such as a processor, an operating system, system memory, memory storage devices, input-output controllers, input-output devices, and display devices. It will also be understood by those of ordinary skill in the relevant art that there are many possible configurations and components of a computer and may also include cache memory, a data backup unit, and many other devices. To provide for interaction with a user, embodiments of the subject matter described in this specification can be implemented on a computer having a display device, e.g., an LCD (liquid crystal display), LED (light emitting diode) display, or OLED (organic light emitting diode) display, for displaying information to the user.

Examples of input devices include a keyboard, cursor control devices (e.g., a mouse or a trackball), a microphone, a scanner, and so forth, wherein the user can provide input to the computer. Other kinds of devices can be used to provide for interaction with a user as well; for example, feedback provided to the user can be in any form of sensory feedback, e.g., visual feedback, auditory feedback, or tactile feedback; and input from the user can be received in any form, including acoustic, speech, or tactile input. Examples of output devices include a display device (e.g., a monitor or projector), speakers, a printer, a network card, and so forth. Display devices may include display devices that provide visual information, this information typically may be logically and/or physically organized as an array of pixels. In addition, a computer can interact with a user by sending documents to and receiving documents from a device that is used by the user; for example, by sending web pages to a web browser on a user's client device in response to requests received from the web browser.

An interface controller may also be included that may comprise any of a variety of known or future software programs for providing input and output interfaces. For example, interfaces may include what are generally referred to as “Graphical User Interfaces” (often referred to as GUI's) that provide one or more graphical representations to a user. Interfaces are typically enabled to accept user inputs using means of selection or input known to those of ordinary skill in the related art. In some implementations, the interface may be a touch screen that can be used to display information and receive input from a user. In the same or alternative embodiments, applications on a computer may employ an interface that includes what are referred to as “command line interfaces” (often referred to as CLI's). CLI's typically provide a text based interaction between an application and a user. Typically, command line interfaces present output and receive input as lines of text through display devices. For example, some implementations may include what are referred to as a “shell” such as Unix Shells known to those of ordinary skill in the related art, or Microsoft® Windows Powershell that employs object-oriented type programming architectures such as the Microsoft®. NET framework.

Those of ordinary skill in the related art will appreciate that interfaces may include one or more GUI's, CLI's or a combination thereof. A processor may include a commercially available processor such as a Celeron, Core, or Pentium processor made by Intel Corporation®, a SPARC processor made by Sun Microsystems®, an Athlon, Sempron, Phenom, or Opteron processor made by AMD Corporation®, or it may be one of other processors that are or will become available. Some embodiments of a processor may include what is referred to as multi-core processor and/or be enabled to employ parallel processing technology in a single or multi-core configuration. For example, a multi-core architecture typically comprises two or more processor “execution cores”. In the present example, each execution core may perform as an independent processor that enables parallel execution of multiple threads. In addition, those of ordinary skill in the related field will appreciate that a processor may be configured in what is generally referred to as 32 or 64 bit architectures, or other architectural configurations now known or that may be developed in the future.

A processor typically executes an operating system, which may be, for example, a Windows type operating system from the Microsoft Corporation®; the Mac OS X operating system from Apple Computer Corp.®; a Unix® or Linux®-type operating system available from many vendors or what is referred to as an open source; another or a future operating system; or some combination thereof. An operating system interfaces with firmware and hardware in a well-known manner, and facilitates the processor in coordinating and executing the functions of various computer programs that may be written in a variety of programming languages. An operating system, typically in cooperation with a processor, coordinates and executes functions of the other components of a computer. An operating system also provides scheduling, input-output control, file and data management, memory management, and communication control and related services, all in accordance with known techniques.

Connecting components may be properly termed as computer-readable media. For example, if code or data is transmitted from a website, server, or other remote source using a coaxial cable, fiber optic cable, twisted pair, digital subscriber line (DSL), or wireless technology such as infrared, radio, or microwave signals, then the coaxial cable, fiber optic cable, twisted pair, DSL, or wireless technology are included in the definition of medium. Combinations of media are also included within the scope of computer-readable media.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

The reference numbers recited in the below claims are solely for ease of examination of this patent application, and are exemplary, and are not intended in any way to limit the scope of the claims to the particular features having the corresponding reference numbers in the drawings.

What is claimed is:

1) A method for remotely supervising preparation and dispensing of medication to a recipient at a remote dispensing unit, the method comprising:
(a) authenticating a recipient identity corresponding to the recipient using a first biometric sensing component;
(b) authenticating, for one or more supervising entities, a supervising identity corresponding to the supervising entity using a second biometric sensing component;
(c) establishing a real-time audiovisual communication session between the recipient at the remote dispensing unit and the one or more supervising entities;
(d) receiving, at a supervisory computing platform, an electronic medication order and label data corresponding to the electronic medication order, originating from an external clinical information system;
(e) authorizing one or more medication doses to be prepared at the remote dispensing unit based on the electronic medication order;
(f) prior to release of a prepared dose, presenting, to the one or more supervising entities, verification data comprising (i) the label data, and (ii) at least one of pump feedback, tablet dispensing feedback, or captured image data of the prepared dose; and
(g) actuating a controlled mechanical transfer mechanism of the remote dispensing unit to release the prepared dose to the recipient only after at least one of the one or more supervisory entities is authenticated and provides an affirmative release authorization.

2) The method of claim 1, wherein both the recipient and the one or more supervisory entities are biometrically authenticated prior to actuation of the controlled mechanical transfer mechanism.

3) The method of claim 1, further comprising enabling participation of a plurality of authenticated session participants in the real-time audiovisual communication session, wherein only participants assigned a supervisory dosing role are permitted to authorize actuation of the controlled mechanical transfer mechanism.

4) The method of claim 1, further comprising, in response to a supervisory command, performing at least one of: cancelling preparing a dose, quarantining the prepared dose, locking a secure access door, unlocking the secure access door, or preventing transfer of the prepared dose to a recipient-accessible compartment.

5) The method of claim 1, wherein the verification data further comprises multiple camera perspectives including at least two of: a recipient view, a preparation-area view, and a transfer-mechanism view.

6) The method of claim 1, wherein determining the recipient identity of the recipient comprises capturing biometric data using at least one of a fingerprint sensor, retina scanner, facial recognition component, or combination thereof.

7) The method of claim 1, wherein the remote dispensing unit comprises a private booth or enclosure configured for the real-time audiovisual communication session.

8) The method of claim 1, wherein the medication comprises a controlled substance.

9) The method of claim 8, wherein the controlled substance comprises methadone, buprenorphine, or both.

10) The method of claim 1, wherein the one or more supervisory entities comprise pharmacists, nurses, or physicians.

11) The method of claim 1, further comprising evaluating recipient eligibility for releasing the prepared dose using an automated decision module configured to analyze recipient responses, biometric verification data, and historical session data, wherein releasing is conditionally authorized when predefined criteria are satisfied and wherein the one or more supervisory entities retain override authority.

12) The method of claim 1, wherein the remote dispensing unit comprises an internal isolation gate configured to prevent insertion of a recipient's hand into a secure preparation compartment during preparing or transferring the prepared dose.

13) A computing system for remotely supervising preparation and dispensing of medication to a recipient at a remote dispensing unit, the computing system comprising:
(a) a communication component configured to communicatively couple to a plurality of remote dispensing units and to an external clinical information system;
(b) a processor configured to execute computer-readable instructions;
(c) a biometric authentication component configured to receive one or more recipient biometric identifiers from the recipient; and
(d) a memory storing computer-readable instructions that, when executed by the processor, cause the computing system to:
(i) receive the one or more recipient biometric identifiers associated with the recipient at the remote dispensing unit and confirm a recipient identity corresponding to the recipient based on the one or more recipient biometric identifiers;
(ii) receive, for one or more supervisory entities, one or more supervisor biometric identifiers associated with the supervising entity and confirm a supervisor identity corresponding to the supervising entity based on the one or more supervisor biometric identifiers;
(iii) establish a real-time audiovisual communication session between the recipient and at least one authenticated session participant;
(iv) receive an electronic medication order and label data corresponding to the electronic medication order, originating from the external clinical information system;
(v) transmit authorization signals to initiate one or more medication doses to be prepared at the remote dispensing unit based on the electronic medication order;
(vi) display verification data to the one or more supervisory entities prior to dose release, the verification data comprising the label data and at least one of pump feedback, tablet dispensing feedback, or captured image data of a prepared dose; and
(vii) transmit a control signal that actuates a controlled mechanical transfer mechanism at the remote dispensing unit to release the prepared dose to the recipient only after at least one of the one or more supervisory entities is authenticated and provides an affirmative release authorization.

14) The computing system of claim 13, wherein the one or more supervisory entities authenticated using both a personal identification credential and a biometric identifier prior to transmission of the control signal that actuates the controlled mechanical transfer mechanism.

15) The computing system of claim 13, wherein the computing system is configured to concurrently supervise and control a plurality of geographically distributed remote dispensing units.

16) The computing system of claim 13, wherein the remote dispensing unit comprises (i) a secure preparation compartment and (ii) a recipient interaction compartment separated by the controlled mechanical transfer mechanism.

17) The computing system of claim 13, wherein the medication comprises a controlled substance, and wherein the controlled substance comprises methadone, buprenorphine, or both; or any other controlled or non-controlled medication.

18) A distributed computing system for remotely supervising preparation and dispensing of medication to a recipient at a remote dispensing unit, the distributed computing system comprising:
(a) a plurality of remote dispensing units, each remote dispensing unit comprising:
(i) a biometric sensing component configured to capture one or more recipient biometric identifiers of the recipient;
(ii) a remote communication component;
(iii) a remote processor; and
(iv) a remote memory storing instructions that cause the remote dispensing unit to transmit the one or more recipient biometric identifiers and verification data to a hub computing system; and
(b) the hub computing system comprising:
(i) a hub communication component configured to communicatively couple to the plurality of remote dispensing units and an external clinical information system;
(ii) a hub processor; and
(iii) a hub memory storing instructions that cause the hub computing system to:
(A) confirm a recipient identity of the recipient based on the one or more recipient biometric identifiers;
(B) confirm, for one or more supervising entities, a supervisor identity based on one or more supervisor biometric identifiers;
(C) receive an electronic medication order and corresponding label data originating from the external clinical information system;
(D) authorize one or more medication doses to be prepared at a selected remote dispensing unit based on the electronic medication order;
(E) present verification data to a supervisory user prior to dose release; and
(F) transmit a control signal to actuate a controlled mechanical transfer mechanism at the selected remote dispensing unit to release a prepared dose to the recipient only after at least one of the one or more supervisory entities is authenticated and provides an affirmative release authorization.

19) The distributed computing system of claim 18, wherein the biometric sensing component comprises at least one of a fingerprint sensor, retina scanner, facial recognition component, or combination thereof.

20) The distributed computing system of claim 18, wherein the one or more supervisory entities comprise pharmacists, nurses, physicians, or a combination thereof.
